(19) United States
(12) Patent Application Publication
(10) Pub. No.: US 2004/0166516 A1
(43) Pub. Date: Aug. 26, 2004

(54) Methods for generating catalytic proteins

(76) Inventors: Markus Kurz, West Newton, MA (US); Peter Lohse, Weston, MA (US)

Correspondence Address: ROPES & GRAY LLP, ONE INTERNATIONAL PLACE, BOSTON, MA, 02110-2624, US

(21) Appl. No.: 10/725,945

(22) Filed: Dec. 1, 2003

Related U.S. Application Data
(63) Continuation of application No. 09/795,037, filed on Feb. 26, 2001.
(60) Provisional application No. 60/184,515, filed on Feb. 24, 2000.

(51) Int. Cl. C12Q 1/68; C12Q 1/37

(52) U.S. Cl. 435/6; 435/23

(57) ABSTRACT

Disclosed herein are novel methods for the generation and identification of catalytic and autoproteolytic proteins using nucleic acid-protein fusion approaches.

### CROSS REFERENCE TO RELATED APPLICATIONS

This application is a continuation of U.S. application Ser. No. 09/795,037, filed Feb. 26, 2001, now pending, which claims the benefit of provisional application, U.S. S.No. 60/184,515, filed Feb. 24, 2000, now abandoned.

### BACKGROUND OF THE INVENTION

In general, the invention relates to screening methods for catalytic proteins.

To generate enzymes with new or improved functions, several fundamentally different approaches have been developed and tested. The rational design of improved biocatalysts requires a profound understanding of catalytic mechanism and molecular structure to alter the enzyme in a productive fashion. In addition to the difficulty in obtaining necessary structural information, rational enzyme design has proven to be a tedious undertaking. Irrational approaches, such as applied molecular evolution approaches, on the other hand, do not require detailed knowledge of the enzyme structure, but rather rely on the generation of extensive numbers of random mutants of existing enzymes, followed by selection or screening for the most powerful variants (see, for example, Skandalis et al., Chem. Biol. 1997, 4:889; Bornscheuer, Angew. Chem. Int. ed. 1998, 37:3105; Arnold, Acc. Chem. Res. 1998, 31:125; Steipe, Curr. Top. Microbiol. Immunol. 1999, 243:55). Yet another approach exploits the diversity of the immune system to select de novo for antibodies that catalyze chemical reactions (Lerner et al., Science 1991, 252:659).

For the necessary generation of molecular diversity in these starting libraries, a number of methods have been devised, such as chemical synthesis of partially randomized genes, random mutagenesis, and molecular breeding (Skandalis et al., Chem. Biol. 1997, 4:889). In order for a given library member to be selectable, its enzymatic activity must be connected to a change in phenotype. Such phenotypes include the survival of a host cell, expression of a marker substance (e.g., a fluorescent protein), modification of the library member, binding of transition state analogues, or chemical modification by reactive substrate analogues.

These methods use procedures performed in vivo, either for selection or screening or for library preparation, severely restricting library size and diversity, and thus the likelihood of isolating a desired compound (as discussed in Roberts, Curr. Opin. Chem. Biol. 1999, 3:268).

### SUMMARY OF THE INVENTION

In general, the present invention features methods for identifying nucleic acid molecules which encode catalytic proteins. In a first aspect, the invention features a method that involves the steps of: (a) providing a candidate catalytic protein fusion molecule, including a candidate catalytic protein linked to both its nucleic acid coding sequence and a substrate; and (b) determining whether the candidate catalytic protein catalyzes a reaction of the substrate by assaying for an alteration in molecular size, charge, or conformation of the fusion molecule, relative to an unreacted fusion molecule, thereby identifying a nucleic acid molecule which encodes a catalytic protein. The alteration in molecular size, charge, or conformation of the reacted fusion molecule may be detected by an alteration in electrophoretic mobility or by column chromatography (for example, by HPLC, FPLC, ion exchange column chromatography, or size exclusion chromatography analysis).

In a related aspect, the invention features another method for identifying a nucleic acid molecule which encodes a catalytic protein, the method involving the steps of: (a) providing a candidate catalytic protein fusion molecule, including a candidate catalytic protein linked to both its nucleic acid coding sequence and a substrate; (b) allowing the candidate catalytic protein to catalyze a reaction of the substrate in solution; (c) contacting the product of step (b) with a capture molecule that has specificity for and binds a reacted fusion molecule, but not an unreacted fusion molecule, the capture molecule being immobilized on a solid support; and (d) detecting the reacted fusion molecule in association with the solid support, thereby identifying a nucleic acid molecule which encodes a catalytic protein. In a preferred embodiment of this method, the substrate, as a result of the reaction, is covalently bonded to an affinity tag, and the capture molecule binds the affinity tag but does not bind an unreacted fusion molecule.

In a third aspect, the invention features yet another method for identifying a nucleic acid molecule which encodes a catalytic protein, the method involving the steps of: (a) providing a candidate catalytic protein fusion molecule, including a candidate catalytic protein linked to both its nucleic acid coding sequence and a substrate, the substrate being covalently bonded to an affinity tag; (b) allowing the candidate catalytic protein to catalyze a reaction of the substrate in solution; (c) contacting the product of step (b) with a capture molecule that is specific for the affinity tag, the capture molecule being immobilized on a solid support; and (d) determining whether the fusion molecule is bound to the solid support, wherein the determination that a fusion molecule is not bound to the solid support identifies a nucleic acid molecule which encodes a catalytic protein. For this method, the solid support is preferably a column or beads and a fusion molecule that does not bind to the column includes a nucleic acid molecule which encodes a catalytic protein.

In a fourth aspect, the invention features a further method for identifying a nucleic acid molecule which encodes a catalytic protein, the method involving the steps of: (a) providing a candidate catalytic protein fusion molecule, including a candidate catalytic protein linked to both its nucleic acid coding sequence and a substrate; (b) allowing the candidate catalytic protein to catalyze a reaction of the substrate in solution in the presence of an affinity tag, the reaction resulting in the covalent attachment of the affinity tag to the fusion molecule; (c) immunoprecipitating the product of step (b) with an antibody that is specific for the affinity tag; and (d) detecting the immunoprecipitation complex, thereby identifying the fusion molecule as having a nucleic acid molecule which encodes a catalytic protein.

In preferred embodiments of various aspects of the invention, the candidate catalytic protein fusion molecule is present in a population of candidate catalytic protein fusion molecules; the substrate is a protein or a nucleic acid (for example, RNA or DNA); the catalytic protein is a ribonuclease, an RNA ligase, an RNA polymerase, a terminal transferase, a reverse transcriptase, or a tRNA synthetase, and the substrate is RNA; the catalytic protein is a deoxyribonuclease, a restriction endonuclease, a DNA ligase, a terminal transferase, a DNA polymerase, or a polynucleotide kinase, and the substrate is DNA; the substrate is covalently bonded to the candidate catalytic protein fusion molecule; the substrate is a substrate-nucleic acid conjugate and the nucleic acid portion of the conjugate is linked to the nucleic acid portion of the candidate catalytic protein fusion molecule; the substrate is a protein and is linked to the protein portion of the candidate catalytic protein fusion molecule; the substrate is non-covalently associated with the candidate catalytic protein fusion (for example, the substrate is covalently bonded to a nucleic acid strand hybridized to the nucleic acid portion of the candidate catalytic fusion molecule); the nucleic acid coding sequence of the candidate catalytic protein fusion molecule is double-stranded; and the determining or detecting step of the method is carried out by assaying the nucleic acid coding sequence of a fragment thereof.

In addition to the above, the general methods of the invention can also be utilized to identify nucleic acid molecules encoding autoproteolytic proteins. In particular, in a first aspect, the invention features a method for identifying a nucleic acid molecule which encodes an autoproteolytic protein, involving the steps of: (a) providing a candidate autoproteolytic protein fusion molecule, including a candidate autoproteolytic protein linked to its nucleic acid coding sequence; and (b) determining whether the candidate autoproteolytic protein catalyzes a self-reaction by assaying for an alteration in molecular size, charge, or conformation of the fusion molecule, relative to an unreacted fusion molecule, thereby identifying a nucleic acid molecule which encodes an autoproteolytic protein. In this method, the alteration in molecular size, charge, or conformation of the reacted fusion molecule may be detected by an alteration in electrophoretic mobility or column chromatography (for example, by HPLC, FPLC, ion exchange column chromatography, or size exclusion chromatography).

In addition, the invention features a related method for identifying a nucleic acid molecule which encodes an autoproteolytic protein, the method involving the steps of: (a) providing a candidate autoproteolytic protein fusion molecule, including a candidate autoproteolytic protein linked to its nucleic acid coding sequence; (b) allowing the candidate autoproteolytic protein to self-react; (c) contacting the product of step (b) with a capture molecule that has specificity for and binds a self-reacted fusion molecule, but not an unreacted fusion molecule, the capture molecule being immobilized on a solid support; and (d) detecting the self-reacted fusion molecule in association with the solid support, thereby identifying a nucleic acid molecule which encodes an autoproteolytic protein.

In yet another related aspect, the invention features a third method for identifying a nucleic acid molecule which encodes an autoproteolytic protein, the method involving the steps of: (a) providing a candidate autoproteolytic protein fusion molecule, including a candidate autoproteolytic protein linked to its nucleic acid coding sequence, the protein being covalently bonded to an affinity tag; (b) allowing the candidate autoproteolytic protein to self-react in solution; (c) contacting the product of step (b) with a capture molecule that is specific for the affinity tag, the capture molecule being immobilized on a solid support; and (d) determining whether the fusion molecule is bound to the solid support, wherein the determination that a fusion molecule not bound to the solid support identifies a nucleic acid molecule which encodes an autoproteolytic protein. In this method, the solid support is a column or beads and a fusion molecule that does not bind to the column includes a nucleic acid molecule which encodes an autoproteolytic protein.

In a fourth approach for identifying a nucleic acid molecule which encodes an autoproteolytic protein, the invention features a method involving the steps of: (a) providing a candidate autoproteolytic protein fusion molecule, including a candidate autoproteolytic protein linked to its nucleic acid coding sequence; (b) allowing the candidate autocatalytic protein to self-react in solution; (c) immunoprecipitating the product of step (b) with an antibody that is specific for a reacted fusion molecule; and (d) detecting the immunoprecipitation complex, thereby identifying the fusion molecule as having a nucleic acid molecule which encodes an autoproteolytic protein.

In preferred embodiments of various aspects of the invention, the candidate autoproteolytic protein fusion molecule is present in a population of candidate autoproteolytic protein fusion molecules; the autoproteolytic protein is a self-cleaving enzyme; the autoproteolytic protein is a self-splicing enzyme; and the nucleic acid coding sequence of the candidate autoproteolytic protein fusion molecule is double-stranded.

As used herein, by a “protein” is meant any two or more naturally occurring or modified amino acids joined by one or more peptide bonds. “Protein” and “peptide” are used interchangeably herein.

By a “nucleic acid” is meant any two or more covalently bonded nucleotides or nucleotide analogs or derivatives. As used herein, this term includes, without limitation, DNA, RNA, and PNA. A “nucleic acid coding sequence” can therefore be DNA (for example, cDNA), RNA, PNA, or a combination thereof. By “DNA” is meant a sequence of two or more covalently bonded, naturally occurring or modified deoxyribonucleotides. By “RNA” is meant a sequence of two or more covalently bonded, naturally occurring or modified ribonucleotides. One example of a modified RNA included within this term is phosphorothioate RNA.

As used herein, by “linked” is meant covalently or non-covalently associated.

By “covalently bonded” to a peptide acceptor is meant that the peptide acceptor is joined to a “protein coding sequence” either directly through a covalent bond or indirectly through another covalently bonded sequence.

By “non-covalently bonded” is meant joined together by means other than a covalent bond (for example, by hybridization).

By a “population” is meant more than one molecule (for example, more than one RNA, DNA, or RNA-protein fusion molecule). Because the methods of the invention facilitate selections which begin, if desired, with large numbers of candidate molecules, a “population” according to the invention preferably means more than 10⁹ molecules, more preferably, more than 10¹¹, 10¹², or 10¹³ molecules, and, most preferably, more than 10¹³ molecules. When present in such a population of molecules, a desired catalytic protein may be selected from other members of the population. As used herein, by “selecting” is meant substantially partitioning a molecule from other molecules in a population. A “selecting” step provides at least a 2-fold, preferably, a 30-fold, more preferably, a 100-fold, and, most preferably, a 1000-fold enrichment of a desired molecule relative to undesired molecules in a population following the selection step. A selection step may be repeated any number of times, and different types of selection steps may be combined in a given approach.

By a “peptide acceptor” is meant any molecule capable of being added to the C-terminus of a growing protein chain by the catalytic activity of the ribosomal peptidyl transferase function. Typically, such molecules contain (i) a nucleotide or nucleotide-like moiety (for example, adenosine or an adenosine analog (di-methylation at the N-6 amino position is acceptable)), (ii) an amino acid or amino acid-like moiety (for example, any of the 20 D- or L-amino acids or any amino acid analog thereof (for example, O-methyl tyrosine or any of the analogs described by Ellman et al., Meth. Enzymol. 202:301, 1991), and (iii) a linkage between the two (for example, an ester, amide, or ketone linkage at the 3′ position or, less preferably, the 2′ position); preferably, this linkage does not significantly perturb the pucker of the ring from the natural ribonucleotide conformation. Peptide acceptors may also possess a nucleophile, which may be, without limitation, an amino group, a hydroxyl group, or a sulfhydryl group. In addition, peptide acceptors may be composed of nucleotide mimetics, amino acid mimetics, or mimetics of the combined nucleotide-amino acid structure.

By a “capture molecule,” as used herein, is meant any molecule which has a specific, covalent or non-covalent affinity for a portion of a desired catalytic protein fusion molecule or an associated “affinity tag.” Examples of capture molecules and their corresponding affinity tags include, without limitation, members of an antigen/antibody pair, protein/inhibitor pair, receptor/ligand pair (for example, a cell surface receptor/ligand pair, such as a hormone receptor/peptide hormone pair), enzyme/substrate pair, lectin/carbohydrate pair, oligomeric or heterooligomeric protein aggregates, DNA binding protein/DNA binding site pair, RNA/protein pair, and nucleic acid duplexes, heteroduplexes, or ligated strands, as well as any molecule which is capable of forming one or more covalent or non-covalent bonds (for example, disulfide bonds) with any portion of a catalytic protein fusion molecule, affinity tag, or moiety added to such molecules (for example, by post-synthetic modification). A preferred capture molecule/affinity tag pair is an avidin-biotin pair (for example, streptavidin-biotin).

By a “solid support” is meant, without limitation, any column (or column material), bead, test tube, microtiter dish, solid particle (for example, agarose or sepharose), microchip (for example, silicon, silicon-glass, or gold chip), or membrane (for example, the membrane of a liposome or vesicle) to which an affinity complex may be bound, either directly or indirectly (for example, through other binding partner intermediates such as other antibodies or Protein A), or in which an affinity complex may be embedded (for example, through a receptor or channel).

### DESCRIPTION OF THE DRAWINGS

FIGS. 1A-1C are diagrams illustrating exemplary nucleic acid-protein selections involving reactive site binding.

FIG. 2 is a diagram illustrating exemplary nucleic acid-protein selections involving enzyme-substrate chimeras.

FIG. 3 is a diagram illustrating exemplary nucleic acid-protein selections involving nuclease activity.

FIG. 4 is a diagram illustrating exemplary nucleic acid-protein selections involving ligase activity.

FIG. 5 is a diagram illustrating exemplary nucleic acid-protein selections involving polymerase or terminal transferase activity.

FIG. 6 is a diagram illustrating exemplary nucleic acid-protein selections involving kinase or tRNA synthetase activity.

FIGS. 7A-7C are diagrams illustrating exemplary methods for substrate attachment.

FIGS. 8 and 9 are diagrams illustrating exemplary nucleic acid-protein selections involving autoproteolytic reactions.

### DETAILED DESCRIPTION

Described herein are improved in vitro selection methods for isolating RNA-protein fusions (termed PROfusion™) and DNA-protein fusions whose peptide or protein components possess novel or improved catalytic activities. These methods may be used for the isolation of novel enzymes with tailor-made activities and substrate specificities from randomized peptide and protein libraries, or for the directed evolution of existing enzymes with improved catalytic features, including, but not limited to, higher catalytic rates, optimized performance under desired reaction conditions (for example, temperature or solvent conditions), higher or altered substrate specificities, modulated cofactor dependence, and engineered allosteric interactions. The methods described herein utilize recently described nucleic acid-protein fusion technology and therefore exploit all of the advantages inherent in this technology with respect to library size and diversity and ease of fusion preparation. The isolation of products is accomplished through direct selection in vitro, allowing the use of libraries of higher complexity than are used in traditional methods based on genetic selections or screening procedures in vivo. Moreover, reaction conditions are not restricted by host cell environments or other complicated or fragile molecular assemblies and thus can be varied over a broader range. Finally, due to the ease of nucleic acid-fusion preparation methods, selections may be carried out significantly more quickly than is practical for conventional techniques.

Nucleic Acid-Protein Fusion Libraries

The starting point for the selection methods described herein is the preparation of suitable nucleic acid-protein fusion libraries. These fusion libraries may include either RNA-protein fusions (U.S. Ser. No. 09/007,005; U.S. Ser. No. 09/247,190; WO 98/31700; Roberts & Szostak, Proc. Natl. Acad. Sci. USA 1997, 94:12297; Roberts, Curr. Opin. Chem. Biol. 1999, 3:268) or DNA-protein fusions (Lohse et al., U.S. Ser. No. 60/110,549; U.S. Ser. No. 09/453,190; U.S. Ser. No. 99/28,472; WO 00/32823). The design of the library depends on the particular application. For selections that refine a particular, existing catalytic activity (e.g., to achieve higher catalytic rates, optimized performance under desired reaction conditions such as particular temperature or solvent conditions, altered substrate specificities, altered cofactor dependence, or engineered allosteric interactions), variations are introduced into the existing enzyme's genetic information. This can be achieved through any standard method, including chemical synthesis of mutagenized gene fragments, mutagenesis by chemical reagents, mutagenic PCR, DNA shuffling, or reproduction in an E. coli mutator strain (as described, for example, in Skandalis et al., Chem. Biol. 1997, 4:889, and references therein). Alternatively, a semi-rational approach may be used in which multiple independent enzyme domains are joined through peptide linkers, leading to a hybrid enzyme (as described, for example, in Béguin, Curr. Opin. Biotech. 1999, 10:336) or a single-chain enzyme (Tang et al., J. Biol. Chem. 1996, 271:15682). If desired, molecular diversity may also be introduced into each of those domains, for example, by the methods described above. If the de novo generation of an enzymatic activity is sought, libraries of proteins or protein scaffolds that are partially or totally randomized may be used. Mutagenesis or randomization is preferably performed at the DNA level (by any standard technique); the resulting gene constructs are used for nucleic acid-protein construction according to previously described standard protocols (for example, (U.S. Ser. No. 09/007,005; U.S. Ser. No. 09/247,190; WO 98/31700; Roberts & Szostak, Proc. Natl. Acad. Sci. USA 1997, 94:12297; U.S. Ser. No. 09/619,103; U.S. Ser. No. 00/19,653; Kurz et al., Nucleic Acids Res. 28:e83, 2000). Depending on the desired in vitro selection method utilized (see below), the fusion molecules may be further modified post-synthetically through the attachment of reactive groups or substrate mimics. To restrict prospective catalytic activity to the protein portion of the fusion, the nucleic acids are preferably rendered catalytically inactive. This may be achieved through generation of a double-stranded nucleic acid (for example, through reverse transcription) prior to the selection step, since catalytic ribozyme and desoxyribozyme structures generally require complex nucleic acid folding which is difficult or impossible or attain as a double-stranded molecule.

Selection Methods

The methods described herein are suitable for directed molecular evolution of known enzymes as well as for selection for de novo enzyme activity, differing mainly in the library utilized. Following function-based selection of a fusion from a library as described below, the fusion may be amplified and propagated, or its genetic information analyzed as described in (U.S. Ser. No. 09/007,005; U.S. Ser. No. 09/247,190; WO 98/31700; Roberts & Szostak, Proc. Natl. Acad. Sci. USA 1997, 94:12297; and Roberts, Curr. Opin. Chem. Biol. 1999, 3:268.

There now follow preferred selection schemes for nucleic acid-protein fusions having desired catalytic functions.

Reactive Site Binding

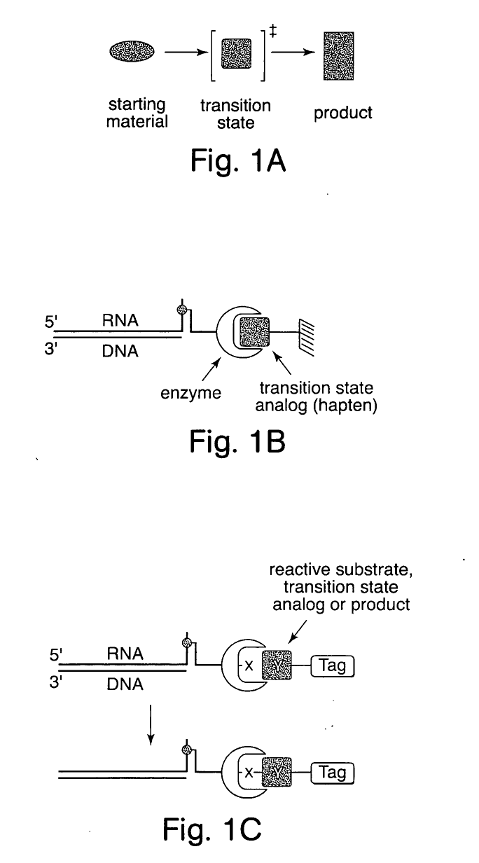

Transition state theory provides that enzymatic activity is governed through stabilization of a reaction's transition state (Jencks, Catalysis in Chemistry and Enzymology, Dover Mineola, N.Y., 1969, Mader & Bartlett, Chem. Rev. 1997, 97:1281) (FIG. 1A). Based on this assumption, nucleic acid-protein fusions may be selected in vitro that bind to suitable hapten molecules that structurally resemble the transition state of a given chemical reaction (FIG. 1B). The selection methodology is essentially the same as previously described for the selection of peptide and protein affinity binders using RNA-protein fusion technology (U.S. Ser. No. 09/007,005; U.S. Ser. No. 09/247,190; WO 98/31700; Roberts & Szostak, Proc. Natl. Acad. Sci. USA 1997, 94:12297; Roberts, Curr. Opin. Chem. Biol. 1999, 3:268). Haptens may be designed as previously described for catalytic antibodies (Lerner et al., Science 1991, 252:659; Fujii et al, Nature Biotech. 1998, 16:463). If desired, a stepwise approach involving the sequential use of various haptens may be utilized to enhance the selection potential (Wentworth Jr., et al., Proc. Natl. Acad. Sci. USA 1998, 95:5971).

In a further variation of the above approach, enzymatically active nucleic acid-protein molecules may be selected using either reactive substrates (Janda et al. Proc. Natl. Acad. Sci. USA 1994, 91:2532; Rahil et al., Bioorg. Med. Chem. 1997, 5:1783; Banzon et al., Biochemistry 1995, 34:743; Vanwetswinkel et al., J. Mol. Biol. 2000, 295:527; Wirsching et al., Science 1995, 270:1775) or products (Janda et al., Science 1997, 275:945) that covalently capture nucleic acid-protein fusions that are capable of substrate binding or catalysis (FIG. 1C).

Use of Enzyme-Substrate Chimeras

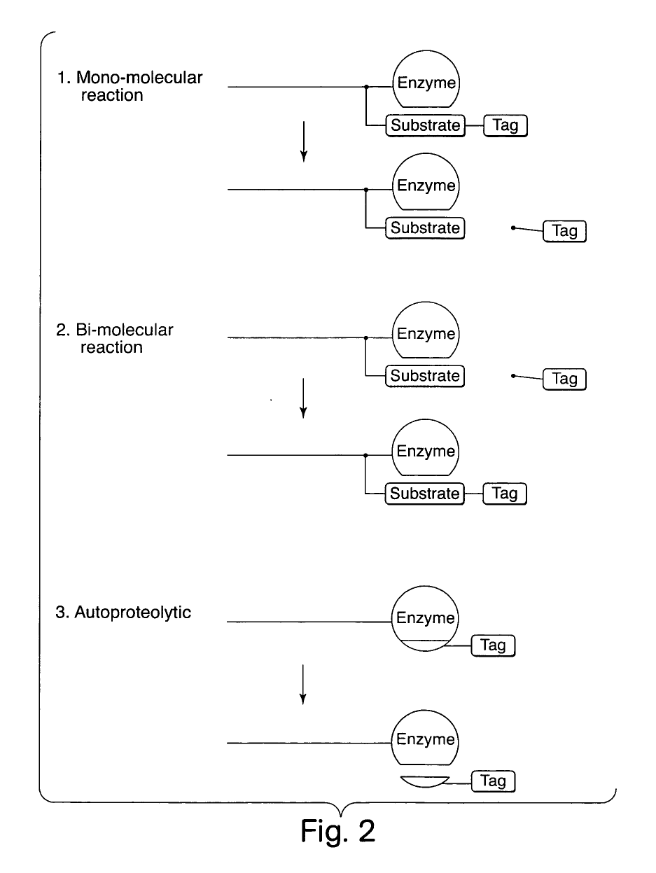

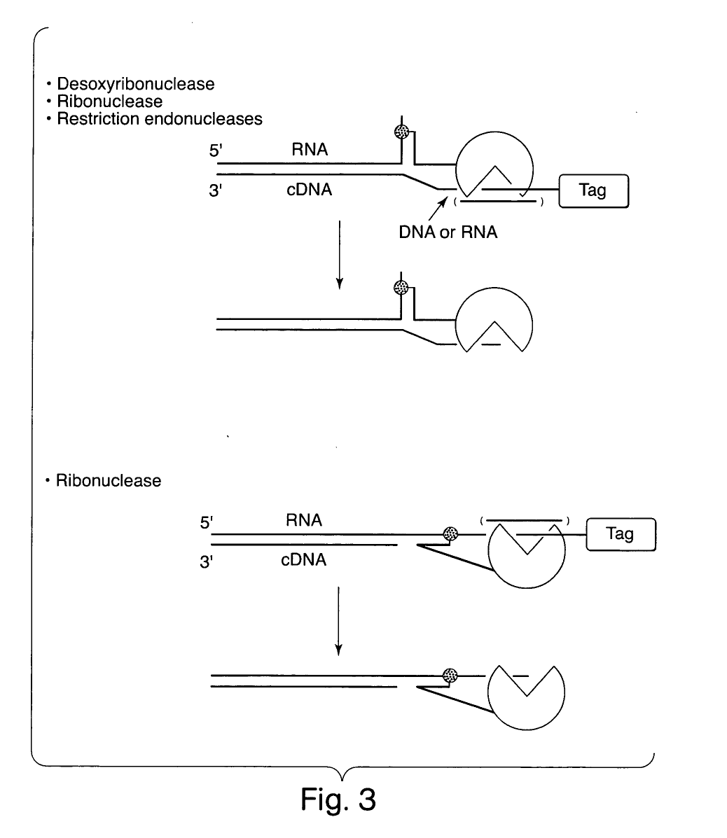

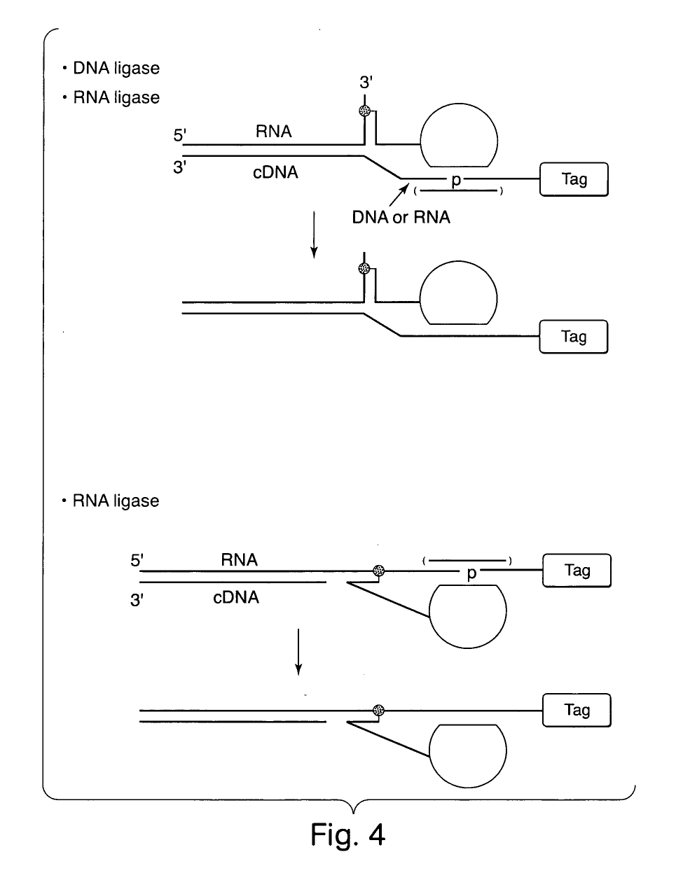

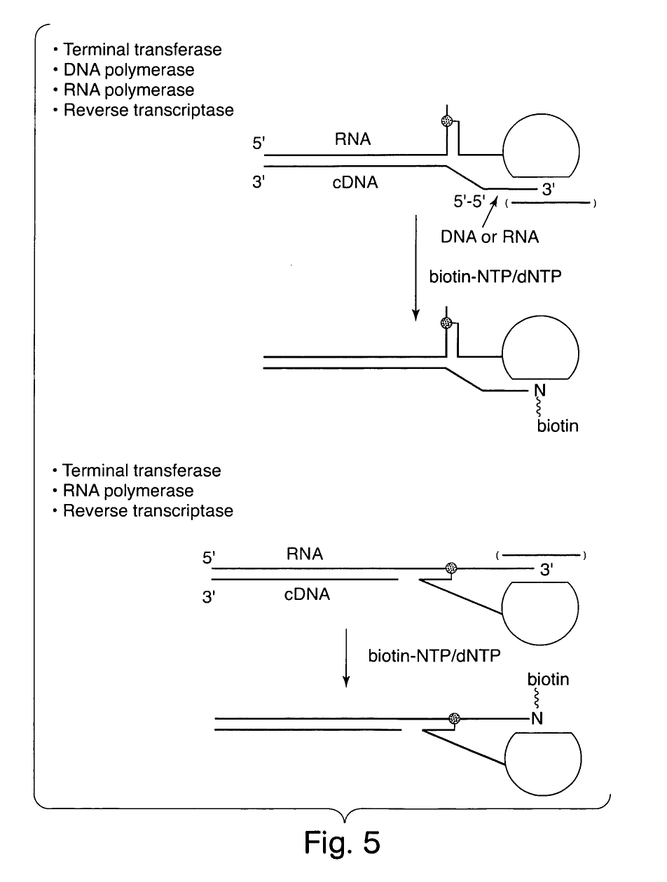

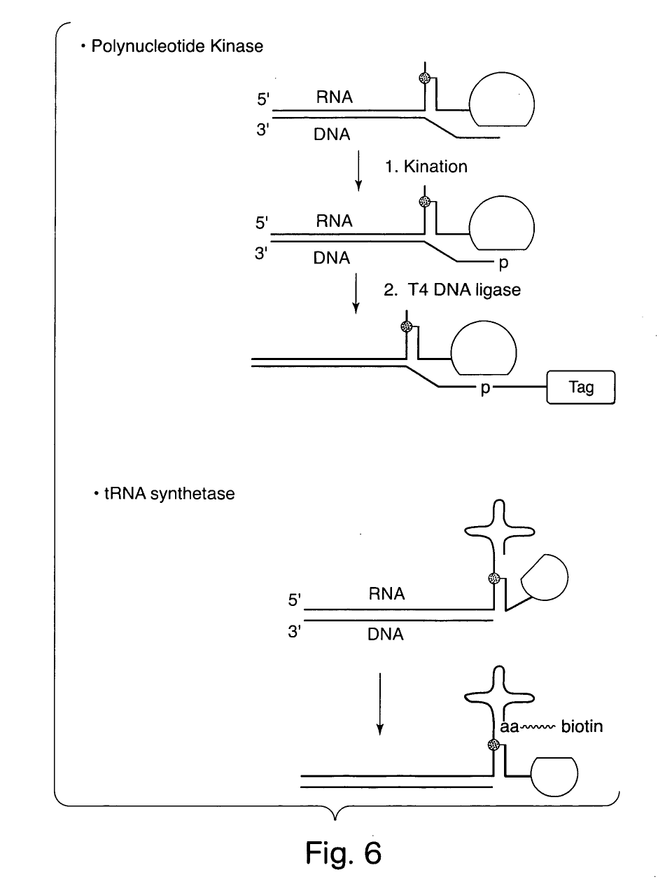

In cases where the catalytic activity of a nucleic acid-protein fusion generates a permanent alteration of its own phenotype, it becomes readily distinguishable from those nucleic acid-protein fusions that do not exhibit a similar enzymatic activity. Favorable self-modifications include the attachment of, or cleavage from, functional units (e.g., biotin) that either allow physical separation of the fusion based on, for example, molecular size, electrophoretic mobility, or affinity capture or retention on a solid phase (FIG. 2) (Pedersen et al., Proc. Natl Acad. Sci. USA 1998, 95:105223; Jestin et al., Angew. Chem. Int. Ed. 1999, 38:1124; Atwell & Wells, Proc. Natl. Acad. Sci. USA 1999, 96:9497). To carry out this technique, a stable connection must be formed between the enzyme nucleic acid-protein fusion and a suitable substrate domain. In one preferred approach, the fusion enzyme domain acts directly on its suitably modified nucleic acid portion. Proposed enzymatic activities include, without limitation, nucleases, ligases, terminal transferase, polynucleotide kinase, tRNA synthetase, and polymerases (see Pedersen et al., Proc. Natl Acad. Sci. USA 1998, 95:105223; Jestin et al., Angew. Chem. Int. Ed. 1999, 38:1124; Sambrook, Fritsch & Maniatis Molecular Cloning, (1989) Cold Spring Harbor Laboratory Press, Cold Spring Harbor) (FIGS. 3-6). Solid phase attachment is most easily achieved through incorporation of binding moieties (for example, biotin moieties) into the nucleic acid substrates or by nucleic acid hybridization to immobilized capture probes. Alternatively, self-modified fusion molecules can be separated after ligation or nucleolytic cleavage from unreacted molecules by gel electrophoretic or chromatographic techniques.

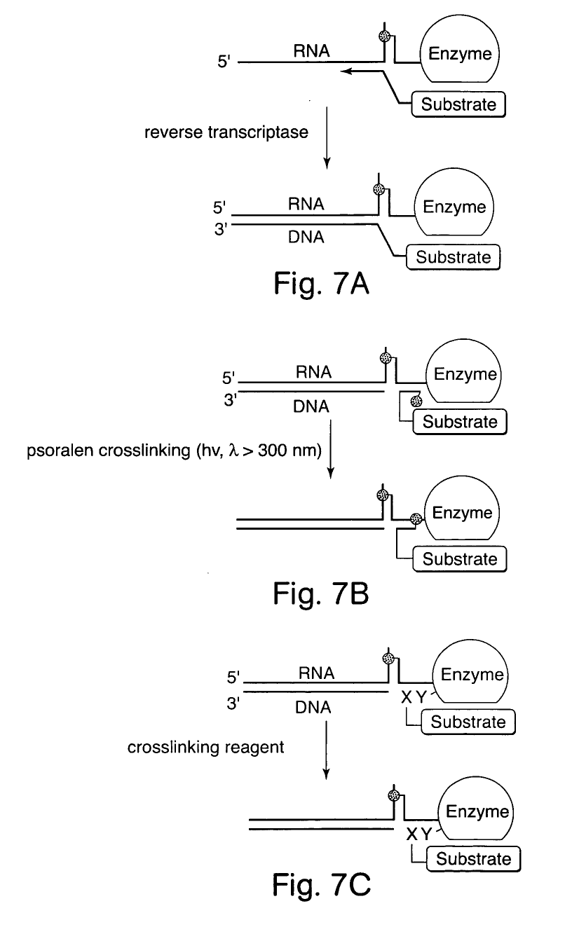

In another approach, substrates (nucleotidic or non-nucleotidic) are connected to the nucleic acid-protein fusion entities. This can be achieved through, for example, the use of suitably modified reverse transcription primers (FIG. 7A), psoralen crosslinking of substrate-nucleic acid conjugates (FIG. 7B; Pieles & Englisch, Nucleic Acids Res 1989, 17:285; Pieles et al., Nucleic Acids Res 1989, 17:8967), or through post-synthetic modification using standard peptide crosslinking agents (FIG. 7C; Pierce Chemical Co., Double-Agents cross-linking reagents selection guide, Rockford, Ill., 1999). Again, the substrates are preferably designed to allow the attachment to, or cleavage from, solid supports or any other alteration that allows physical separation based on, for example, molecular size, electrophoretic mobility, etc, upon enzymatic action (FIG. 2; Atwell & Wells, Proc. Natl. Acad. Sci. USA 1999, 96:9497). This can most easily be achieved through the use of an affinity reagent, such as biotin, tethered to the substrate in a suitable fashion. Alternatively, if a specific antibody is available that recognizes the product structure, the fusion may be isolated by immunoprecipitation.

As for the substrates, the use of any combination of peptides, nucleotides, and small organic molecules is possible, depending on the goal of the particular selection. The tether which connects the substrate moieties to the fusion should preferably be chosen such that it allows unrestricted access to the fusion's enzymatic core, and is therefore preferably constructed from flexible linker units, such as alkyl- or polyethylene glycol chains.

If a self-cleavage reaction is desired, the enzyme activity may be controlled by the choice of reaction medium or cofactor. This allows controlled fusion synthesis under conditions that suppress catalytic activity. For example, following immobilization and washes, enzyme activity may be switched on by supplying the appropriate medium, leading to release of catalytically active fusion molecules.

Preferably, the substrate domains are covalently attached to the fusion's cDNA portion. This eliminates the requirement to isolate or select the entire fusion molecule after enzymatic reaction, but allows the retrieval of the cDNA only. This is particularly useful when using denaturing gel-electrophoresis to partition unreacted from reacted fusions based on differences in size or electrophoretic mobility.

Autoproteolytic Reactions

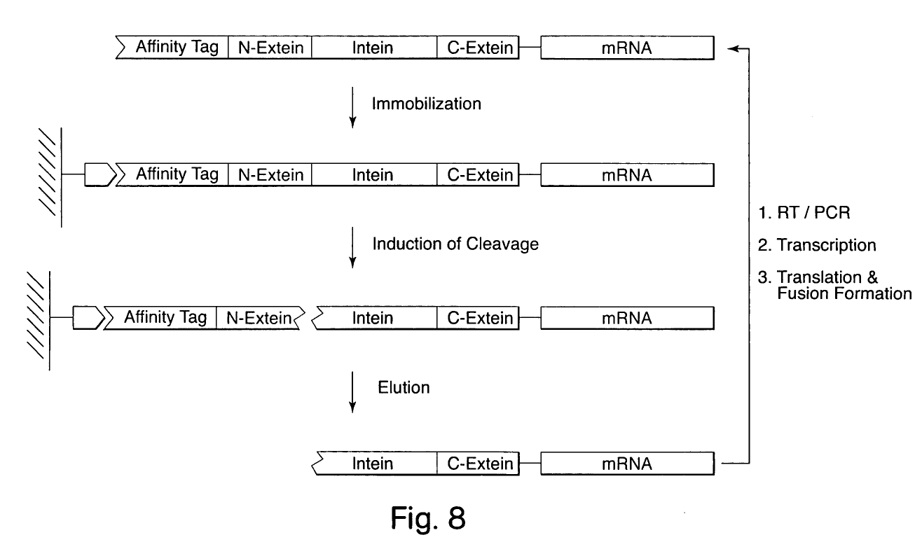

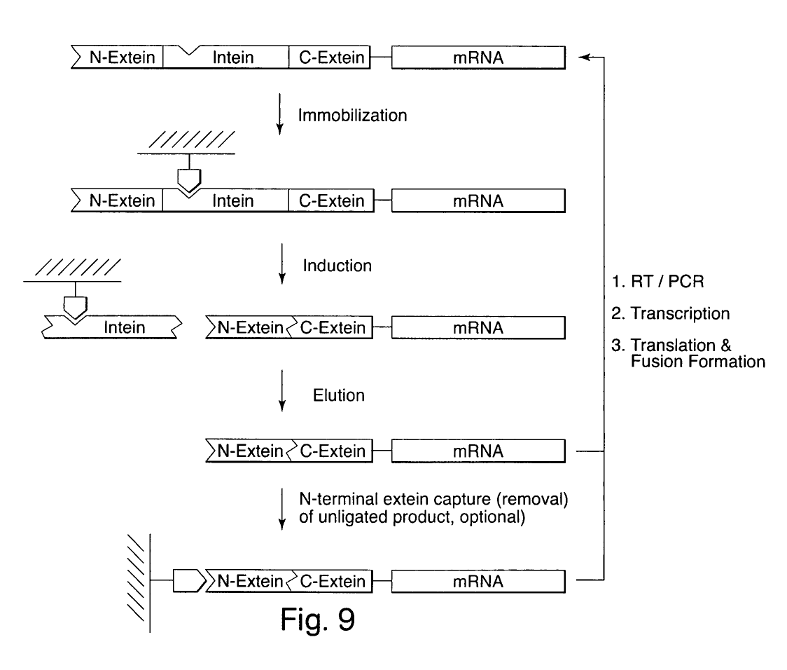

A third class of potential catalytic activities involves protein splicing and related autoproteolytic reactions (Perler et. al., Curr. Opin. Chem. Biol. 1997, 1:292). In one preferred approach, nucleic acid-protein fusion molecules are constructed that contain an N-terminal affinity tag, followed by a suitable (randomized) intein sequence. After immobilization through the affinity tag, self-cleavage is induced through supply of the desired reaction medium or cofactor, and the C-terminal cleavage fragment (including the nucleic acid portion) is recovered and amplified (FIG. 8). In a variant of this approach, the affinity tag is included in the intein region. After excision of the intein, followed by extein ligation, the products are released from the solid phase and recovered (FIG. 9). If extein ligation is an essential feature of the product, an additional affinity purification step against the N-terminal extein portion may be included.

Alternatively, cleaved or spliced fusion molecules may be separated from uncleaved or unspliced fusion molecules by molecular size (for example, by gel electrophoresis).

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

What is claimed is:

1. A method for identifying a nucleic acid molecule which encodes a catalytic protein, said method comprising the steps of:
a) providing a candidate catalytic protein fusion molecule, comprising a candidate catalytic protein linked to both its nucleic acid coding sequence and a substrate; and
b) determining whether said candidate catalytic protein catalyzes a reaction of said substrate by assaying for an alteration in molecular size, charge, or conformation of said fusion molecule, relative to an unreacted fusion molecule, thereby identifying a nucleic acid molecule which encodes a catalytic protein.

2. The method of claim 1, wherein said alteration in molecular size, charge, or conformation of said reacted fusion molecule is detected by an alteration in electrophoretic mobility.

3. The method of claim 1, wherein said alteration in molecular size, charge, or conformation of said reacted fusion molecule is detected by column chromatography.

4. The method of claim 3, wherein said alteration in molecular size, charge, or conformation of said reacted fusion molecule is detected by HPLC, FPLC, ion exchange column chromatography, or size exclusion chromatography.

5. A method for identifying a nucleic acid molecule which encodes a catalytic protein, said method comprising the steps of:
a) providing a candidate catalytic protein fusion molecule, comprising a candidate catalytic protein linked to both its nucleic acid coding sequence and a substrate;
b) allowing said candidate catalytic protein to catalyze a reaction of said substrate in solution;
c) contacting the product of step (b) with a capture molecule that has specificity for and binds a reacted fusion molecule, but not an unreacted fusion molecule, said capture molecule being immobilized on a solid support; and
d) detecting said reacted fusion molecule in association with said solid support,
thereby identifying a nucleic acid molecule which encodes a catalytic protein.

6. The method of claim 6, wherein, as a result of said reaction, said substrate is covalently bonded to an affinity tag and said capture molecule binds said affinity tag but does not bind an unreacted fusion molecule.

7. A method for identifying a nucleic acid molecule which encodes a catalytic protein, said method comprising the steps of:
a) providing a candidate catalytic protein fusion molecule, comprising a candidate catalytic protein linked to both its nucleic acid coding sequence and a substrate, said substrate being covalently bonded to an affinity tag;
b) allowing said candidate catalytic protein to catalyze a reaction of said substrate in solution;
c) contacting the product of step (b) with a capture molecule that is specific for said affinity tag, said capture molecule being immobilized on a solid support; and
d) determining whether said fusion molecule is bound to said solid support, wherein the determination that a fusion molecule is not bound to said solid support identifies a nucleic acid molecule which encodes a catalytic protein.

8. The method of claim 7, wherein said solid support is a column or beads and a fusion molecule that does not bind to said column includes a nucleic acid molecule which encodes a catalytic protein.

9. A method for identifying a nucleic acid molecule which encodes a catalytic protein, said method comprising the steps of:
a) providing a candidate catalytic protein fusion molecule, comprising a candidate catalytic protein linked to both its nucleic acid coding sequence and a substrate;
b) allowing said candidate catalytic protein to catalyze a reaction of said substrate in solution in the presence of an affinity tag, said reaction resulting in the covalent attachment of said affinity tag to said fusion molecule;
c) immunoprecipitating the product of step (b) with an antibody that is specific for said affinity tag; and
d) detecting said immunoprecipitation complex, thereby identifying said fusion molecule as having a nucleic acid molecule which encodes a catalytic protein.

10. The method of claim 1, 5, 7, or 9, wherein said candidate catalytic protein fusion molecule is present in a population of candidate catalytic protein fusion molecules.

11. The method of claim 1, 5, 7, or 9, wherein said substrate is a protein.

12. The method of claim 1, 5, 7, or 9, wherein said substrate is a nucleic acid.

13. The method of claim 12, wherein said nucleic acid is RNA.

14. The method of claim 1 or 7, wherein said catalytic protein is a ribonuclease and said substrate is RNA.

15. The method of claim 1, 5, or 9, wherein said catalytic protein is an RNA ligase, an RNA polymerase, a terminal transferase, a reverse transcriptase, or a tRNA synthetase and said substrate is RNA.

16. The method of claim 12, wherein nucleic acid is DNA.

17. The method of claim 1 or 7, wherein said catalytic protein is a deoxyribonuclease or a restriction endonuclease and said substrate is DNA.

18. The method of claim 1, 5, or 9, wherein said catalytic protein is a DNA ligase, a terminal transferase, a DNA polymerase, or a polynucleotide kinase and said substrate is DNA.

19. The method of claim 1, 5, or 9, wherein said substrate is covalently bonded to said candidate catalytic protein fusion molecule.

20. The method of claim 7 or 19, wherein said substrate is a substrate-nucleic acid conjugate and the nucleic acid portion of said conjugate is linked to the nucleic acid portion of said candidate catalytic protein fusion molecule.

21. The method of claim 7 or 19, wherein said substrate is a protein and is linked to the protein portion of said candidate catalytic protein fusion molecule.

22. The method of claim 1, 5, or 9, wherein said substrate is non-covalently associated with said candidate catalytic protein fusion molecule.

23. The method of claim 22, wherein said substrate is covalently bonded to a nucleic acid strand hybridized to the nucleic acid portion of said candidate catalytic fusion molecule.

24. The method of claim 1, 5, 7, or 9, wherein said nucleic acid coding sequence of said candidate catalytic protein fusion molecule is double-stranded.

25. The method of claim 1, wherein, in step (b), said determining step is carried out by assaying for an alteration in molecular size, charge, or conformation of the nucleic acid coding sequence of a fragment thereof.

26. The method of claim 5, wherein, in step (d), said detecting step is carried out by detecting the nucleic acid coding sequence or a fragment thereof in association with said solid support.

27. The method of claim 7, wherein, in step (d), said determining step is carried out by determining whether or not the nucleic acid coding sequence or a fragment thereof is bound to said solid support.

28. The method of claim 9, wherein, in step (d), said detecting step is carried out by detecting the nucleic acid coding sequence or a fragment thereof in said immunoprecipitation complex.

29. A method for identifying a nucleic acid molecule which encodes an autoproteolytic protein, said method comprising the steps of:
a) providing a candidate autoproteolytic protein fusion molecule, comprising a candidate autoproteolytic protein linked to its nucleic acid coding sequence; and
b) determining whether said candidate autoproteolytic protein catalyzes a self-reaction by assaying for an alteration in molecular size, charge, or conformation of said fusion molecule, relative to an unreacted fusion molecule, thereby identifying a nucleic acid molecule which encodes an autoproteolytic protein.

30. The method of claim 29, wherein said alteration in molecular size, charge, or conformation of said reacted fusion molecule is detected by an alteration in electrophoretic mobility.

31. The method of claim 29, wherein said alteration in molecular size, charge, or conformation of said reacted fusion molecule is detected by column chromatography.

32. The method of claim 31, wherein said alteration in molecular size, charge, or conformation of said reacted fusion molecule is detected by HPLC, FPLC, ion exchange column chromatography, or size exclusion chromatography.

33. A method for identifying a nucleic acid molecule which encodes an autoproteolytic protein, said method comprising the steps of:
a) providing a candidate autoproteolytic protein fusion molecule, comprising a candidate autoproteolytic protein linked to its nucleic acid coding sequence;
b) allowing said candidate autoproteolytic protein to self-react;
c) contacting the product of step (b) with a capture molecule that has specificity for and binds a self-reacted fusion molecule, but not an unreacted fusion molecule, said capture molecule being immobilized on a solid support; and
d) detecting said self-reacted fusion molecule in association with said solid support, thereby identifying a nucleic acid molecule which encodes an autoproteolytic protein.

34. A method for identifying a nucleic acid molecule which encodes an autoproteolytic protein, said method comprising the steps of:
a) providing a candidate autoproteolytic protein fusion molecule, comprising a candidate autoproteolytic protein linked to its nucleic acid coding sequence, said protein being covalently bonded to an affinity tag;
b) allowing said candidate autoproteolytic protein to self-react in solution;
c) contacting the product of step (b) with a capture molecule that is specific for said affinity tag, said capture molecule being immobilized on a solid support; and
d) determining whether said fusion molecule is bound to said solid support, wherein the determination that a fusion molecule not bound to said solid support identifies a nucleic acid molecule which encodes an autoproteolytic protein.

35. The method of claim 34, wherein said solid support is a column or beads and a fusion molecule that does not bind to said column includes a nucleic acid molecule which encodes an autoproteolytic protein.

36. A method for identifying a nucleic acid molecule which encodes an autoproteolytic protein, said method comprising the steps of:
a) providing a candidate autoproteolytic protein fusion molecule, comprising a candidate autoproteolytic protein linked to its nucleic acid coding sequence;
b) allowing said candidate autocatalytic protein to self-react in solution;
c) immunoprecipitating the product of step (b) with an antibody that is specific for a reacted fusion molecule; and
d) detecting said immunoprecipitation complex, thereby identifying said fusion molecule as having a nucleic acid molecule which encodes an autoproteolytic protein.

37. The method of claim 29, 33, 34, or 36, wherein said candidate autoproteolytic protein fusion molecule is present in a population of candidate autoproteolytic protein fusion molecules.

38. The method of claim 29, 33, 34, or 36, wherein said autoproteolytic protein is a self-cleaving enzyme.

39. The method of claim 29, 33, 34 or 36, wherein said autoproteolytic protein is a self-splicing enzyme.

40. The method of claim 29, 33, 34, or 36, wherein said nucleic acid coding sequence of said candidate autoproteolytic protein fusion molecule is double-stranded.
